(19) United States
(12) Patent Application Publication
(10) Pub. No.: US 2024/0132863 A1
(43) Pub. Date: Apr. 25, 2024

(54) DNASE 1-LIKE 2 ENGINEERED FOR MANUFACTURING AND USE IN THERAPY

(71) Applicant: Neutrolis, Inc, Cambridge, MA (US)

(72) Inventors: Tobias A. FUCHS, Cambridge, MA (US); Angelene Prasanna, Cambridge, MA (US)

(21) Appl. No.: 18/546,864

(22) PCT Filed: Feb. 17, 2022
(86) PCT No.: PCT/US22/16771

§ 371 Date: Aug. 16, 2023

Related U.S. Application Data
(60) Provisional application No. 63/150,433, filed on Feb. 17, 2021.

(51) Int. Cl. C12N 9/22; C07K 14/765

(52) U.S. Cl. CPC C12N 9/22; C07K 14/765; C12Y 301/21; A61K 38/00; C07K 2319/02; C07K 2319/31

(57) ABSTRACT

The present disclosure provides a D1L2 enzyme lacking a proline-rich domain (PRD) and/or lacking the C-terminal Arg residue, which is efficiently produced in microbial expression systems such as Pichia pastoris, and has high activity for degrading chromatin at acidic pH. The present disclosure provides methods of the production and use of the D1L2 enzyme in therapy.

### PRIORITY

This application claims the benefit of, and priority to, U.S. Provisional Application No. 63/150,433, filed Feb. 17, 2021, the entire contents of which are hereby incorporated by reference.

### BACKGROUND

Inflammation is an essential host response to control invading microbes and heal damaged tissues. Uncontrolled and persistent inflammation causes tissue injury in a plethora of inflammatory disorders. Neutrophils are the predominant leukocytes in acute inflammation. During infections neutrophils generate neutrophil extracellular traps (NETs), lattices of DNA-filaments decorated with toxic histones and enzymes that immobilize and neutralize bacteria. However, excessive NET formation may harm host cells due to their cytotoxic, proinflammatory, prothrombotic activity, and pro-tumorigenic activity.

DNASE1-LIKE 2 (also referred to herein as DNASE1L2 or D1L2), belongs to the DNASE1 protein family, a homologous group of secreted endonuclease enzymes that can degrade extracellular DNA. D1L2 is the only divalent cation-dependent acidic DNase in mammals. Thus, D1L2 has unique features that are likely physiologically and therapeutically relevant. While D1L2 may have desirable enzymatic properties for treating or controlling inflammatory conditions or infection of various tissues, e.g., by degrading extracellular DNA, D1L2 has not been described as suitable for manufacturing using conventional recombinant systems.

Accordingly, the invention provides recombinant D1L2 enzymes with improvements for manufacturing and therapy, as well as methods for producing the recombinant D1L2 enzyme and using the recombinant D1L2 enzyme for therapy. The present disclosure demonstrates that the recombinant D1L2 enzyme has high chromatin-degrading activity, particularly at acidic pH.

### SUMMARY OF DISCLOSURE

The present disclosure is based, in part, on the discovery that a D1L2 enzyme lacking a proline-rich domain (PRD) and/or lacking the C-terminal Arg residue is efficiently produced in microbial expression systems and has high activity for degrading chromatin at acidic pH.

In one aspect, the disclosure provides a recombinant D1L2 enzyme lacking a PRD domain, and/or lacking a C-terminal Arginine that is naturally present in D1L2 isoform 1 and isoform 2. The D1L2 enzyme according to this aspect has high recombinant expression properties, including in non-mammalian expression systems such as Pichia pastoris. Accordingly, in aspects of the disclosure, a method is provided for producing recombinant D1L2 enzyme lacking a PRD and/or a C-terminal Arginine. The method comprises culturing a non-mammalian host cell encoding the D1L2 enzyme and recovering the enzyme from the culture. An exemplary D1L2 enzyme in accordance with the disclosure is represented by SEQ ID NO: 7.

In various embodiments, the D1L2 gene that is expressed in the expression system encodes signal peptide that is essentially native to a wild-type human DNase. In some embodiments, the signal peptide is from human D1 (SEQ ID NO: 13), D1L2 (SEQ ID NO: 8), D1L1 (SEQ ID NO: 14), or D1L3 (SEQ ID NO: 15). In some embodiments, the signal peptide is a non-native (to the D1L2 enzyme) signal peptide suitable for secretion from host cells of the non-mammalian expression system. An exemplary non-native signal peptide is α-Mating Factor (SEQ ID NO: 9), which may be used for example, with yeast expression systems such as Pichia pastoris.

In some embodiments, the D1L2 enzyme is fused to a carrier protein, optionally by means of an amino acid linker. The carrier protein can be an albumin amino acid sequence. Such fusion to a carrier protein can provide benefits in in vivo persistence, as well as in recombinant expression.

In various embodiments in accordance with the disclosure, the recombinant D1L2 enzyme is not glycosylated. In various embodiments, the D1L2 enzyme produced according to this disclosure is substantially non-immunogenic.

In some embodiments, the D1L2 enzyme is engineered to have physical, pharmacodynamics, and/or enzymatic properties suitable for therapy, including therapy using topical or locally administered compositions. In various embodiments, the engineered D1L2 enzyme may have the same or higher protein-free DNA (naked DNA) degrading activity, the same or higher chromosomal DNA (chromatin) degrading activity, similar or improved protease resistance, and higher production levels in non-mammalian expression systems (such as Pichia pastoris) than D1L2 isoform 1 (defined by SEQ ID NO: 1 or SEQ ID NO: 4).

In various embodiments, the D1L2 enzyme contains one or more polyethylene glycol (PEG) moieties or other conjugates (e.g., fatty acid or acyl groups). In some embodiments, one or more PEGylated amino acids are selected from lysine, cysteine, histidine, arginine, aspartic acid, glutamic acid, serine, threonine and tyrosine. In some embodiments, one or more amino acids suitable for PEGylation are introduced by substitution of one or more amino acids in D1L2 (e.g., in SEQ ID NO: 7). In some embodiments, one or more PEGylated amino acids are cysteine (Cys or C) and PEGylation is conducted via thiol conjugation. In some embodiments, NETS-PEG reagent can be used to PEGylate primary amines in D1L2.

According to this disclosure, the D1L2 enzyme is encoded by a polynucleotide, which can be codon optimized for expression in the non-mammalian expression system. In some embodiments, the polynucleotide comprises a plasmid vector. In various embodiments, the plasmid vector comprises a nucleic acid encoding the D1L2 enzyme or variant described herein, operably linked to an expression control region that is functional in the host cell. The expression control region is capable of driving expression of the operably linked encoding nucleic acid such that the enzyme is produced in a cell transformed with the expression vector. In other aspects, the disclosure provides a non-mammalian expression cell (a “host cell”), which comprises the expression vector encoding the D1L2 enzyme described herein. In some embodiments, the cell is a yeast cell, such as Pichia pastoris.

In still other aspects, the disclosure provides pharmaceutical compositions for topical or local administration. The compositions comprise recombinant D1L2 enzyme described herein, or as produced according to this disclosure, and a pharmaceutically acceptable carrier.

In some embodiments, the pharmaceutical composition is formulated for topical administration, such as a cream, lotion, gel, solution, foam, spray, ointment, mouthwash, shampoo or soap. In some embodiments, the pharmaceutical composition is for local or topical treatment of a tissue having an acidic environment and/or biolfilms. For example, the acidic environment may have a pH of about 6.0 or less, about 5.5 or less, or about 5.0 or less, or about 4.5 or less, or about 4.0 or less. In some embodiments, the composition is a skin care composition for maintaining healthy skin and attractive appearance of skin. In these embodiments, the composition is useful in a method of treating or preventing infection and/or inflammation of the skin. In some embodiments employing a topical composition, the subject may have a condition that impacts the appearance or health of skin, hair, or nails. In some embodiments, the subject has a condition such as psoriasis, dermatitis, epidermolysis bullosa, or an allergic reaction (e.g., comprising hives or rash). In some embodiments, the subject has dry skin, such as xeroderma. In some embodiments, the subject has acne vulgaris. In still other embodiments, the subject's skin may be characterized by overgrowth of Staphylococcus aureus, which can be associated with atopic dermatitis. In some embodiments, the subject has psoriasis, and the composition is applied to psoriatic lesions.

In still other embodiments, the composition is formulated for administration to one or more of the eyes, ears, nasal or sinus cavity, and oral cavity. Compositions according to these embodiments can be useful in a method for treating or preventing inflammation or infection of the eyes of a subject, for example, where the composition is formulated as an eye drop or eye wash (including an eye lid cleaner). In some embodiments, the subject may have dry eye disease, or may have allergic, bacterial, or viral conjunctivitis. Bacterial conjunctivitis can be exacerbated by biofilm formation. In some embodiments, the subject has inflamed ducts of the eye, as may manifest as blepharitis or related condition (e.g., chalazion). In still other embodiments, the composition can be useful for treating or preventing inflammation or infection of the ears of a subject, for example, where the composition formulated as an ear drop. In still other embodiments, the composition can be useful in a method for treating or preventing of nasal or sinus inflammation or infection of a subject, for example, where the composition is formulated as a nasal spray. In some embodiments, the subject has a sinus infection or recurring sinus infection. In still other embodiments, the subject has an infection or inflammation of the oral cavity, including gingivitis and dental abscess.

In some embodiments, the composition is useful in a method for the treatment of a nucleic acid-related eye disease. Such diseases include dry eye disease, lamellar keratitis, contact lens-associated keratitis, endophthalmitis, infectious crystalline keratopathy, ocular cicatricial pemphigoid (OCP), keratoconjunctivitis sicca (KCS), Sjogren syndrome (SS), Sjogren syndrome associated keratoconjunctivitis sicca, non-Sjogren syndrome associated keratoconjunctivitis sicca, keratitis sicca, sicca syndrome, xerophthalmia, tear film disorder, decreased tear production, aqueous tear deficiency (ATD), and meibomian gland dysfunction (MGD).

In still other embodiments, the composition is formulated for pulmonary delivery, such as a powder or solution aerosol. In some embodiments, the composition is formulated for delivery by nebulizer. In various embodiments, the composition is useful in a method for treating or preventing inflammation or infection of the lungs of a patient. In some embodiments, the subject has cystic fibrosis, pneumonia, bronchitis, asthma, lower respiratory tract infection, acute respiratory distress syndrome (ARDS), acute lung injury (ALI), transfusion-induced lung injury (TRALI), or chronic obstructive pulmonary disease (COPD). In some embodiments, the subject has pulmonary fibrosis (e.g., idiopathic pulmonary fibrosis, or IPF). In still other embodiments, the subject has a chronic or recurring pulmonary infection.

In the case of cystic fibrosis, it is difficult to eradicate or control Pseudomonas aeruginosa infection, which is partially due to the forming of biofilms. By administering the D1L2 enzyme in accordance with this disclosure, for example, by nebulizer, it may be possible to control Pseudomonas infection in cystic fibrosis patients and relieve some overuse of antibiotics. In various embodiments, the D1L2 enzyme provided herein has higher activity in CF lung secretions than PULMOZYME (dornase alfa).

In still other embodiments, the patient may have an indwelling medical device, which tend to favor microbial colonization and biofilm formation. In some embodiments, the D1L2 enzyme or composition comprising the D1L2 enzyme is administered to a patient on mechanical ventilation to avoid or reduce biofilm formation.

Other aspects and embodiments of this disclosure will be apparent from the following detailed description.

### BRIEF DESCRIPTION OF THE DRAWINGS

FIG. 1 shows an amino acid sequence alignment of human DNASE1L2 isoform 1 (DNASE1L2-L, SEQ ID NO: 1, Uniprot identifier: Q92874-1) and the human DNASE1L2 isoform 2 (DNASE1L2-S; SEQ ID NO: 2, Uniprot identifier: Q92874-2). The secretory signal peptide (SEQ ID NO: 8) and proline rich domain (PRD, SEQ ID NO: 3) are highlighted. Sequence rulers indicate the amino acid position. The mature forms of isoform 1 (SEQ ID NO: 4) and isoform 2 (SEQ ID NO: 6) are included.

FIG. 2 shows that deletion of the proline-rich domain increases DNASE1L2 expression in Pichia pastoris. Shown are the estimated protein concentrations in supernatants of the three best performing strains, calculated by peak area for bovine serum albumin diluted in mock strain matrix at known concentrations. Expression constructs include (a) isoform 1 (SEQ ID NO: 4) with native signal peptide (SP, SEQ ID NO: 8), (b) isoform 1 (SEQ ID NO: 4) with alpha mating factor (a1VIF, SEQ ID NO: 9), and (c) isoform 2 (SEQ ID NO: 6) with alpha mating factor (aMF, SEQ ID NO: 9).

FIG. 3 shows that purified proline-rich domain deleted DNASE1L2 (PRDD-DNASE1L2) degrades chromatin at acidic pH. Nuclei, as a source of high molecular weight chromatin, were incubated with purified PRDD-DNASE1L2 at pH levels ranging from 3.5 to 7.5. Degradation of high molecular weight to low molecular weight was observed at acidic pH 3.5 to 5.5. Basic-domain deleted (BDD)-DNASE1L3, which served as a control, showed degradation at neutral pH 6.5 and 7.5.

FIG. 4 shows an alignment of DNASE1L2 amino acid sequences of different species extracted from the Uniprot database. The alignment indicates that only DNASE1L2 from humans, chimpanzees, and baboons features a C-terminal arginine residue.

FIG. 5 shows that deletion of the C-terminal arginine residue increases DNASE1L2 isoform 2 expression in Pichia pastoris. Shown are the estimated protein concentrations in supernatants of the three best performing strains, calculated by peak area for bovine serum albumin diluted in mock strain matrix at known concentrations. Expression constructs include (a) isoform 1 (SEQ ID NO: 4), (b) isoform 2 (SEQ ID NO: 6), and (c) isoform 2 lacking the C-terminal arginine residue (de1278R in SEQ ID NO: 6) generating a new variant (SEQ ID NO: 7). All constructs employed the alpha mating factor (aMF, SEQ ID NO: 9) as a secretory signal peptide.

FIG. 6 shows D1L2 (PRDD) expressed in Pichia pastoris as a fusion with albumin at the N-terminus.

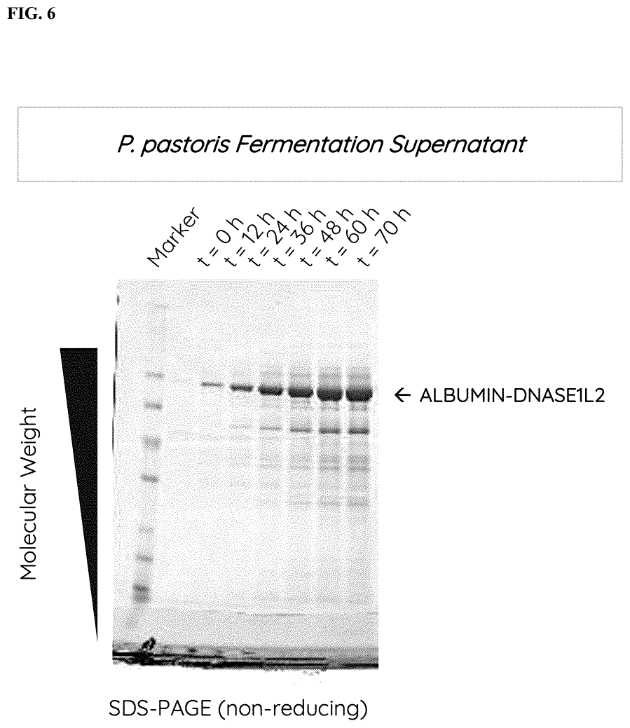

### DETAILED DESCRIPTION

The present disclosure is based, in part, on the discovery that a D1L2 enzyme lacking a proline-rich domain (PRD), and/or lacking the C-terminal Arg residue, is efficiently produced in microbial strains and has high activity for degrading chromatin at acidic pH.

DNASE1L2 (or D1L2) is expressed and secreted in two isoforms, SEQ ID NO: 1 and SEQ ID NO: 2, which differ in the presence or absence of the PRD, respectively. The isoform 1 is expressed in terminally differentiated keratinocytes of the human epidermis, hair follicles and the nail matrix. Knockdown of D1L2 in a human in vitro skin model suppressed the degradation of nuclear DNA and caused retention of nuclei in the stratum corneum, a condition known as parakeratosis. Fischer H. et al., DNASE1L2 degrades nuclear DNA during corneocyte formation. J Invest Dermatol. 2007 Jan; 127(1):24-30. Parakeratotic lesions of skin diseases such as psoriasis can be associated with reduced D1L2 expression. Further, D1L2 can suppress biofilm formation of pathogens such as Pseudomonas aeruginosa and Staphylococcus aureus, among others. Eckhart L, et al., DNASE1L2 suppresses biofilm formation by Pseudomonas aeruginosa and Staphylococcus aureus. Br J. Dermatol. 2007 156(6):1342-5. Isoform 2 is expressed in leukocytes under inflammatory conditions, but the functions are poorly characterized. Shiokawa et al. Characterization of the human DNASE1L2 gene and the molecular mechanism for its transcriptional activation induced by inflammatory cytokines. Genomics. 2004 84:95-105.

While D1L2 may have desirable enzymatic properties for treating or controlling inflammatory conditions or infection of various tissues, e.g., by degrading extracellular DNA, D1L2 has not been described as suitable for manufacturing using conventional recombinant systems. Accordingly, the present disclosure, in the various aspects and embodiments, provides recombinant D1L2 enzymes and methods for producing recombinant D1L2 enzymes, including for use in therapy. In the various embodiments, the invention provides for high expression of the D1L2 enzyme in non-mammalian expression systems, such as yeast expression systems (e.g., Pichia pastoris), the D1L2 enzyme having robust activity for therapeutic applications. In particular, the D1L2 enzyme has robust activity at acidic pH (e.g., <about 6.0).

In one aspect, the disclosure provides a method for producing recombinant D1L2 enzyme. The method comprises culturing a non-mammalian host cell encoding a D1L2 enzyme substantially lacking the Proline Rich Domain (PRD) defined by SEQ ID NO: 3. In various embodiments, the D1L2 produced by the host cell will comprise a signal peptide directing secretion from the cell, which is generally removed during secretion. The method further comprises recovering the D1L2 enzyme from the culture media.

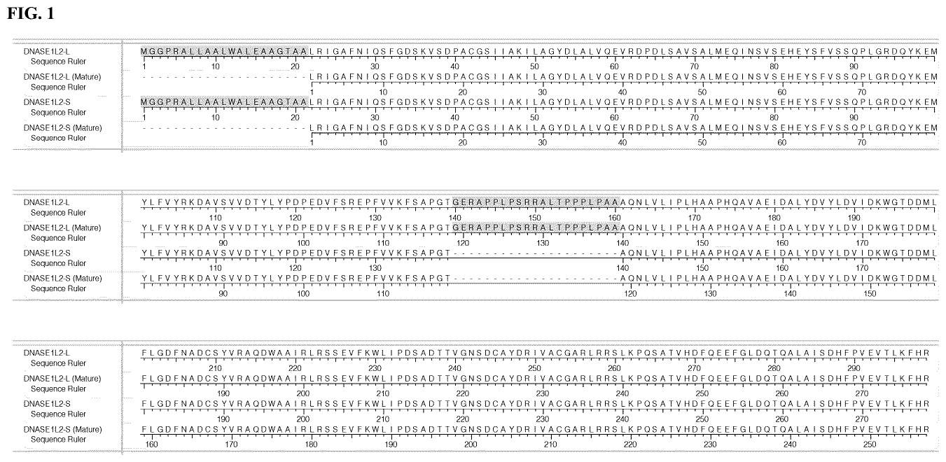

Human D1L2-isoform 1 contains a 21-amino acid PRD, as illustrated in FIG. 1. Removal of this PRD (as in isoform 2) substantially improves expression in non-mammalian expression systems. Exemplary expression systems include eukaryotic expression systems such as yeast expression systems, as well as insect cell or plant cell expression systems. An exemplary non-mammalian expression system is Pichia pastoris. Suitable non-mammalian expression systems include, but are not limited to, as insect cell-based expression systems such as Sf9 cells and baculovirus-based vectors; bacterial expression systems such as Escherichia coli, Lactoccocus lactis, Pseudomonas fluorescens and Bacillus subtilis; yeast and filamentous fungal expression systems such as Aspergillus spp., Saccharomyces cerevisiae, Pichia pastoris and Yarrowia hpolytica; plant-based expression systems such as Nicotiana benthamiana and Nicotiana tabacum. See e.g., U.S. Pat. Nos. 10,385,319; 10,899,801; 10,287,555; 9,931,390; 9,879,280; 9,458,487; 8,535,930; 8,067,198; 7,229,792; 6,558,920; 5,637,477, which are hereby incorporated by reference in its entirety.

In various embodiments, the D1L2 enzyme lacks at least about 10 amino acids of the PRD (SEQ ID NO: 3). In some embodiments, the D1L2 enzyme lacks at least about 15 amino acids, or at least about 18 amino acids of the PRD (SEQ ID NO: 3), or lacks the full PRD. In some embodiments, the PRD is replaced with an alternative sequence that has low or no more than minimal homology to the PRD, such as less than about 8 or less than about 5 identical amino acids that align at a corresponding position. In some embodiments, the alternative sequence has no more than about three, or no more than about two, or no more than one proline residue. In some embodiments, the D1L2 enzyme having a deleted or modified PRD comprises an amino acid sequence having at least about 80% sequence identity to the enzyme defined by SEQ ID NO: 4 (Isoform 1) or SEQ ID NO: 6 (Isoform 2). In some embodiments, the D1L2 enzyme comprises an amino acid sequence having at least about 85% sequence identity, or at least about 90% sequence identity to the enzyme defined by SEQ ID NO: 4 or SEQ ID NO: 6, or at least about 95% sequence identity to the enzyme defined by SEQ ID NO: 4 or SEQ ID NO: 6, or at least about 97% sequence identity to the enzyme defined by SEQ ID NO: 4 or SEQ ID NO: 6.

In some embodiments, the D1L2 enzyme comprises an amino acid sequence having at least about 85% sequence identity, or at least about 90% sequence identity to the enzyme defined by SEQ ID NO: 6, or at least about 95% sequence identity to the enzyme defined by SEQ ID NO: 6, or at least about 97% sequence identity to the enzyme defined by SEQ ID SEQ ID NO: 6.

Alternatively or in addition, the disclosure provides a recombinant D1L2 enzyme lacking a C-terminal Arginine that is naturally present in D1L2 isoform 1 and isoform 2. The D1L2 enzyme according to this aspect has high recombinant expression properties, including in non-mammalian expression systems such as Pichia pastoris. Accordingly, in aspects of the disclosure, a method is provided for producing recombinant D1L2 enzyme lacking a C-terminal Arginine. The method comprises culturing a non-mammalian host cell encoding a D1L2 enzyme lacking the C-terminal Arg residue. Non-limiting examples of D1L2 enzymes lacking the C-terminal Arg residue are SEQ ID NO: 5 and SEQ ID NO: 7. In some embodiments, the D1L2 enzyme that lacks the C-terminal Arg residue comprises an amino acid sequence having at least about 80% sequence identity to the enzyme defined by SEQ ID NO: 5 or SEQ ID NO: 7. In some embodiments, the D1L2 enzyme that lacks the C-terminal Arg residue comprises an amino acid sequence having at least about 85% sequence identity, or at least about 90% sequence identity to the enzyme defined by SEQ ID NO: 5 or SEQ ID NO: 7, or at least about 95% sequence identity to the enzyme defined by SEQ ID NO: 5 or SEQ ID NO: 7, or at least about 97% sequence identity to the enzyme defined by SEQ ID NO: 5 or SEQ ID NO: 7. In various embodiments, the D1L2 enzyme produced by the host cell will comprise a signal peptide directing secretion from the cell, which is generally removed during secretion. The method further comprises recovering the D1L2 enzyme from the culture media.

In some embodiments, the D1L2 enzyme has a deleted or modified PRD (as described), as well as a truncation of the C-terminal Arg. In embodiments, the D1L2 enzyme that substantially lacks the Proline Rich Domain (PRD) and lacks the C-terminal Arg residue comprises an amino acid sequence having at least about 80% sequence identity to the enzyme defined by SEQ ID NO: 7. In some embodiments, the D1L2 that substantially lacks the Proline Rich Domain (PRD) and lacks the C-terminal Arg residue comprises an amino acid sequence having at least about 85% sequence identity, or at least about 90% sequence identity to the enzyme defined by SEQ ID NO: 7, or at least about 95% sequence identity to the enzyme defined by SEQ ID NO: 7, or at least about 97% sequence identity to the enzyme defined by SEQ ID NO: 7.

As used herein, when referring to sequence identity between DNase enzymes, and unless stated otherwise, sequences refer to mature enzymes lacking the signal peptide. Further, unless stated otherwise, amino acid positions are numbered with respect to the full translated DNase sequence, including signal peptide, for clarity.

The similarity of nucleotide and amino acid sequences, i.e. the percentage of sequence identity, can be determined via sequence alignments as known in the art. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). Exemplary algorithms are incorporated into the BLASTN and BLASTP programs of Altschul et al (1990) J. Mol. Biol. 215: 403-410. When using BLAST programs, the default parameters of the respective programs are used.

In various embodiments, the encoded signal peptide is essentially native to a wild-type human DNase. In some embodiments, the signal peptide is from human D1 (SEQ ID NO: 13), D1L2 (SEQ ID NO: 8), D1L1 (SEQ ID NO: 14), or D1L3 (SEQ ID NO: 15). In some embodiments, one, two, or three amino acid substitutions are made to the wild-type signal sequence to enhance production or secretion from the non-mammalian expression system. In some embodiments, the signal peptide is a non-native (to the D1L2 enzyme) signal peptide suitable for secretion from host cells of the non-mammalian expression system. An exemplary non-native signal peptide is α-Mating Factor (SEQ ID NO: 9), which may be used for example, with yeast expression systems such as Pichia pastoris. In some embodiments, the D1L2 enzyme is expressed using an albumin secretory signal peptide as exemplified by SEQ ID NO: 10.

In various embodiments, the non-mammalian expression system (e.g., Pichia pastoris) co-expresses or overexpresses one or more genes that facilitate signal peptide processing. In some embodiments, the gene encodes a protease having an activity for cleavage of signal peptide from secreted proteins. An exemplary protease is KEX2.

In some embodiments, the D1L2 enzyme is fused to a carrier protein, optionally by means of an amino acid linker. The carrier protein can be a polypeptide, such as albumin, transferrin, an Fc domain, XTEN (see U.S. Pat. No. 8,492,530 which is hereby incorporated by reference in its entirety), or elastin-like protein. See, e.g., U.S. Pat. No. 9,458,218, which is hereby incorporated by reference in its entirety. Such fusion can provide benefits in in vivo persistence, as well as in recombinant expression.

In some embodiments, the carrier protein is an albumin amino acid sequence, and the albumin amino acid sequence is fused at the N-terminus of the D1L2, optionally with an interposed linker. An exemplary albumin amino acid sequence is provided by SEQ ID NO: 11. In some embodiments, the albumin amino acid sequence or domain of the fusion protein has at least about 75%, or at least about 80%, or at least about 85%, at least 90%, at least 95%, at least 97%, at least 98%, or at least 99% sequence identity to the reference albumin sequence defined by SEQ ID NO: 11. In some embodiments, the albumin amino acid sequence or domain comprises or consists of the reference albumin sequence defined by SEQ ID NO: 11. In various embodiments, the albumin amino acid sequence binds to the neonatal Fc receptor (FcRn), e.g., human FcRn. The albumin amino acid sequence may be a variant of wild-type HSA (e.g., as represented by SEQ ID NO: 11). In various embodiments, albumin variants may have from one to twenty, or from one to ten amino acid modifications independently selected from deletions, substitutions, and insertions with respect to SEQ ID NO: 11. In some embodiments, the albumin amino acid sequence is any mammalian albumin amino acid sequence. Various modification to the albumin sequence that enhance its ability to serve as a carrier are known, and such modifications can be employed with the present invention. Exemplary modifications to the albumin amino acid sequence are described in U.S. Pat. Nos. 8,748,380, 10,233,228, 10,208,102, and 10,501,524, which are each hereby incorporated by reference in their entireties. Exemplary modifications include one or more (or all) of E505Q, T527M, and K573P as described therein.

In some embodiments, the albumin amino acid sequence or domain is a fragment of full-length albumin, as represented by SEQ ID NO: 11. The term “fragment,” when used in the context of albumin, refers to any fragment of full-length albumin or a variant thereof (as described above) that improves expression of a D1L2 enzyme to which it is fused or conjugated, relative to the corresponding non-fused D1L2 (e.g., in Pichia pastoris). In some embodiments, a fragment of an albumin can refer to an amino acid sequence comprising a fusion of multiple domains of albumin (see, e.g., WO2011/124718), such as domains I and III, and domains II and III. Generally, a fragment of albumin has at least about 100 amino acids or at least about 200 or at least about 300 amino acids of the full-length sequence. In various embodiments, the albumin fragment maintains the ability to bind human FcRn.

In some embodiments, the D1L2 enzyme is fused to a carrier polypeptide (such as an albumin amino acid sequence) through a peptide linker. The peptide linker may be a flexible linker, a rigid linker, or in some embodiments a physiologically-cleavable linker (e.g., a protease-cleavable linker). In some embodiments, the linker is 5 to 100 amino acids in length, or is 5 to 50 amino acids in length. In some embodiments, the linker is from about 10 to about 35 amino acids in length, or from about 15 to about 35 amino acids. In some embodiments, the linker is a flexible linker of from 20 to 40 amino acids. Flexible linkers can comprise predominately (or consist of) Gly and Ser amino acid residues.

In some embodiments, the D1L2 enzyme is fused to an Fc domain. See, for example, WO 2005047334A1, WO 2004074455A2, US 20070269449, which are hereby incorporated by reference in their entirety. In some embodiments, the human Fc domain is selected from IgG1, IgG2, IgG3, and IgG4. In some embodiments, the human Fc domain is a human IgG Fc domain. In some embodiments, the Fc domain has at least two heavy chain constant region domains (CH2 and CH3) and a hinge region. Fc domain can be joined to the D1L2, optionally with an interposed linker, at the N-terminus and/or the C-terminus of the D1L2 enzyme. In some embodiments, the D1L2 enzyme comprises an Fc domain sequence fused to the N-terminus of the mature D1L2 enzyme with an interposed amino acid linker. The peptide linker may be a flexible linker, a rigid linker, or in some embodiments a physiologically-cleavable linker. In some embodiments, the D1L2 enzyme comprises an Fc domain sequence fused to the C-terminus of the D1L2 enzyme, optionally through a linker (e.g., a flexible linker).

Flexible linkers are predominately or entirely composed of small, non-polar or polar residues such as Gly, Ser and Thr. An exemplary flexible linker comprises (Gly_{y}Ser)ₙS_{z} linkers, where y is from 1 to 10 (e.g., from 1 to 5), n is from 1 to about 10, and z is 0 or 1. In some embodiments, n is from 3 to about 8, or from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 3 to 6). Generally, linkers can be predominately composed of amino acids selected from Gly, Ser, Thr, Ala, and Pro. Exemplary linker sequences contain at least 10 amino acids, and may be in the range of 10 to about 50 amino acids, or about 15 to about 40 amino acids, or about 15 to about 35 amino acids. An exemplary linker design is provided as SEQ ID NO: 12.

In some embodiments, the D1L2 enzyme comprises a linker, wherein the amino acid sequence of the linker is predominately glycine and serine residues, or consists essentially of glycine and serine residues. In some embodiments, the ratio of Ser and Gly in the linker is, respectively, from about 1:1 to about 1:10, from about 1:2 to about 1:6, or about 1:4. Exemplary linker sequences comprise or consist of S(GGS)₄GSS, S(GGS)₉GSS, (GGS)₉GSS. In some embodiments, the linker has at least 10 amino acids, or at least 15 amino acids, or at least 20 amino acids, or at least 25 amino acids, or at least 30 amino acids. For example, the linker may have a length of from 15 to 40 amino acids. In various embodiments, longer linkers of at least 15 amino acids can provide improvements in yield upon expression in Pichia pastoris.

In other embodiments, the linker is a physiologically-cleavable linker, such as a protease-cleavable linker. For example, the protease may be a coagulation pathway protease, such as activated Factor XII. In some embodiments, the linker comprises the amino acid sequence of Factor XI and/or prekallikrein or a physiologically cleavable fragment thereof In other embodiments, the linker includes a peptide sequence that is targeted for cleavage by a neutrophil specific protease, such as neutrophil elastase, cathepsin G, and proteinase 3.

In exemplary embodiments, production of the D1L2 enzyme is conducted with large scale (e.g., batch) fermentation of at least about 1000 L, or at least about 5000 L, or at least about 10,000 L, or at least about 50,000 L. In various embodiments, the fermentation results in at least about 200 mg/L of the D1L2 enzyme. In some embodiments, the fermentation results in at least about 300 mg/L of the D1L2 enzyme, at least about 400 mg/L of the D1L2 enzyme, at least about 600 mg/L of the D1L2 enzyme, or at least about 800 mg/L of the D1L2 enzyme, or at least about 1 g/L of the D1L2 enzyme, or at least about 1.2 g/L of the D1L2 enzyme. In still other embodiments, the fermentation results in at least about 1.5 g/L of the D1L2, or at least about 2 g/L of the D1L2 enzyme, or at least about 5 g/L, or at least about 10 g/L of the D1L2 enzyme.

In various embodiments in accordance with the disclosure, the recombinant D1L2 enzyme is not glycosylated. In various embodiments, the D1L2 enzyme produced according to this disclosure is substantially non-immunogenic.

According to other aspects, this disclosure provides a recombinant D1L2 enzyme produced according to the method described herein. In some embodiments, the D1L2 enzyme is engineered to have physical, pharmacodynamics, and/or enzymatic properties suitable for therapy, including therapy using topical compositions. In various embodiments, the engineered D1L2 enzyme may have the same or higher protein-free DNA (naked DNA) degrading activity, the same or higher chromosomal DNA (chromatin) degrading activity, similar or improved protease resistance, and higher production levels in non-mammalian expression systems (such as Pichia pastoris) than D1L2 isoform 1 (defined by SEQ ID NO: 1 or SEQ ID NO: 4).

In some embodiments, the invention employs a D1L2 enzyme produced by transfer of a single amino acid or multiple-adjacent amino acids, termed “building block”, between two members of the DNasel -protein family, to thereby generate enzymatically active variants of D1L2. A “building block” is defined by amino acids that are variable between two or more members of the DNase1-protein family. These variable amino acids are flanked by amino acids that are conserved between two or more members of the DNase1-protein family (“anchors”). The variable single amino acid or multiple contiguous amino acids (“building blocks”) are exchanged between members of the DNase1-protein family by implanting them between conserved single amino acid or multiple contiguous amino acids (“anchors”). See U.S. Pat. No. 10,696,956, which is hereby incorporated by reference in its entirety.

Where three or more amino acids are transferred, up to 1/3 of the amino acids may be further substituted. For example, where three or six amino acids are transferred as a building block, one or up to two resides may be further substituted, respectively. In some embodiments, four or more amino acids are transferred as a building block substitution, and up to 25% of the transferred amino acids are further substituted, e.g., with conservative or non-conservative amino acid modifications. For example, where four, eight, or twelve amino acids are transferred, one, two, or three amino acids (respectively) may be further substituted in the building block substitution.

In some embodiments, the building block substitutions to D1L2 are selected from (e.g., 1, 2, 3, 4, or 5 selected from) non-human D1L2 proteins and which result in variants of human D1L2 that feature one or more of the following mutations corresponding to SEQ ID NO: 1: L22K, I24V, I29V, S35N, S35H, S35R, S35T, V37A, S38L, A41D, A41V, A41G, G431, 544G, S44i, I45V, K48Q, L55I, L55V, A56T, A56M, P64A, S70D, S70T, A71T, A71L, A71S, A71V, M73L, E74Q, N77H, S78R, E81K, E81R, E83N, S85G, S85N, Q90E, Q90K, Q96H, F103Y, V1041, K107D, A109V, A109T, A109K, V110A, V113L, V113M, D1145, D114E, L117Q, P1195, E122G, V124A, V124F, S126N, E128D, F134V, A136V, A136T, G1385, G138R, T1395, T139C, S148C, A151P, P154A, A159P, A160G, A161P, A161T, Q162D, Q162K, Q162R, Q162T, N163K, N163E, L164V, L164F, I167V, H174N, Q175H, A178T, A178V, D192N, G195N, T1965, D198V, M199L, M1991, S210K, R213K, Q215H, A218P, A2195, E226Q, V2271, S243T, A252V, C253S, A255S, A255V, R256H, L257M, R259K, S260T, L261V, Q264H, T267S, T267A, D270N, G276D, G276S, T280S, T280D, T280A, A284C, I286V, L295F, F297S, F297T, F297P, H298R, and R299del.

Variants of D1L2 according to the building block technology are disclosed in WO 2020/0271953 and WO 2020/163264, which are hereby incorporated by reference in its entirety.

The engineered variants of D1L2 enzyme may comprise one or more amino acid substitutions, additions (insertions), deletions, or truncations in the amino acid sequence of human D1L2 (SEQ ID NO: 7). Amino acid substitutions may include conservative and/or non-conservative substitutions. For example, “conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. “Conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. As used herein, “non-conservative substitutions” are defined as exchanges of an amino acid by another amino acid listed in a different group of the six standard amino acid groups (1) to (6) shown above.

DNA fragmentation of D1L2 and variants can be measured using methods known to those skilled in the art such as gel electrophoresis and DNase activity assays. Methods for quantifying DNA fragmentation activity are described in, for example, U.S. Pat. No. 10,696,956, which is hereby incorporated by reference in its entirety.

According to this disclosure, the D1L2 enzyme is encoded by a polynucleotide, which can be codon optimized for expression in the non-mammalian expression system (including but not limited to Pichia pastoris). In some embodiments, the polynucleotide comprises a plasmid vector. In various embodiments, the plasmid vector comprises a nucleic acid encoding the D1L2 enzyme or variant described herein, operably linked to an expression control region that is functional in the host cell. The expression control region is capable of driving expression of the operably linked encoding nucleic acid such that the enzyme is produced in a cell transformed with the expression vector.

Expression control regions are regulatory polynucleotides (sometimes referred to herein as elements), such as promoters and enhancers, that influence expression of an operably linked nucleic acid. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In other aspects, the disclosure provides a non-mammalian expression cell (i.e., a “host cell”), as described above, which comprises the expression vector encoding the D1L2 enzyme described herein. In some embodiments, the cell is a yeast cell, such as Pichia pastoris.

In various embodiments, the D1L2 enzyme contains one or more polyethylene glycol (PEG) moieties or other conjugates such as fatty acid or acyl conjugates. In some embodiments, one or more PEGylated amino acids are selected from lysine, cysteine, histidine, arginine, aspartic acid, glutamic acid, serine, threonine and tyrosine. In some embodiments, one or more amino acids suitable for PEGylation are introduced by substitution of one or more amino acids in D1L2. In some embodiments, one or more PEGylated amino acids are lysine or arginine, and PEGylation is conducted via amine conjugation. In some embodiments, one or more PEGylated amino acids are glutamine (Gln or Q) and PEGylation is conducted via transglutaminase (TGase) mediated enzymatic conjugation. In some embodiments, one or more PEGylated amino acids are cysteine (Cys or C) and PEGylation is conducted via thiol conjugation. For example, NETS-PEG reagent can be used to modify primary amines in D1L2. In some embodiments, one or more PEGylated amino acids are conjugated with PEG moieties that are independently selected from a linear or branched PEG having molecular weights that are independently selected and in the range of about 2 kDa to about 60 kDa. In some embodiments, the PEG moieties have molecular weights that are independently selected from the range of about 5 kDa to about 60 kDa, or about 5 kDa to about 40 kDa, or about 5 kDa to about 30 kDa, or about 10 kDa to about 60 kDa, or about 10 kDa to about 40 kDa, or about 10 kDa to about 30 kDa.

In some embodiments, the recombinant expression system has a deletion or inactivation of one or more proteases that cleave at paired basic amino acids. Exemplary enzymes include Aspartic proteinase 3 (Ysp1) and Kexin (Kex2) expressed by Pichia pastoris or other yeast systems. In some embodiments, these enzymes are not genetically deleted or inactivated, but their activity is inhibited with a protease inhibitor during recombinant protein production.

Further, in some embodiments, the growth medium for the non-mammalian expression system is supplemented with polyanions such as dextran sulfate, heparins, ferric citrate, and EDTA. In further embodiments, the growth medium is supplemented with dextran sulfate that has an average molecular weight of between 5 kDa and 100 kDa. In some embodiments, the dextran sulfate has an average molecular weight that is about 10 kDa or less, or about 20 kDa or less, or about 30 kDa or less, or about 40 kDa or less, or about 50 kDa or less, or about 75 kDa or less, or about 100 kDa or less. In various embodiments, the polyanion is added to the culture in an amount sufficient to complex with the recombinant protein produced. In some embodiments, the recombinant D1L2 enzyme or variants thereof are purified from the culture medium through a method that includes the dissociation of the enzyme from polyanions such as dextran sulfate, heparins, EDTA. In certain embodiments, the purification method includes strong anion exchange resins such as triethylaminoethyl.

In still other aspects, the disclosure provides pharmaceutical compositions for topical or local administration. The compositions comprise recombinant D1L2 enzyme described herein, or as produced according to this disclosure, and a pharmaceutically acceptable carrier.

In some embodiments, the pharmaceutical composition is formulated for topical administration, such as a cream, lotion, gel, solution, foam, spray, ointment, mouthwash, shampoo or soap. In some embodiments, the pharmaceutical composition is for local or topical treatment of a tissue having an acidic environment and/or biolfilms. For example, the acidic environment may have a pH of about 6.0 or less, about 5.5 or less, or about 5.0 or less, or about 4.5 or less, or about 4.0 or less. In some embodiments, the composition is a skin care composition for maintaining healthy skin and attractive appearance of skin. In these embodiments, the composition is useful in a method of treating or preventing infection and/or inflammation of the skin. For example, in some embodiments, the composition is applied to skin comprising paraketotic lesions. Parakeratosis is a mode of keratinization characterized by the retention of nuclei in the stratum corneum. In the skin, this process leads to the abnormal replacement of annular squames with nucleated cells. Parakeratosis is associated with the thinning or loss of the granular layer and is usually seen in diseases of increased cell turnover, whether inflammatory or neoplastic. Parakeratosis is seen in the plaques of psoriasis and in dandruff.

In some embodiments employing a topical composition, the subject may have a condition that impacts the appearance or health of skin, hair, or nails. In some embodiments, the subject has a condition such as psoriasis, dermatitis, epidermolysis bullosa, or an allergic reaction (e.g., comprising hives or rash). In some embodiments, the subject has dry skin, such as xeroderma. In some embodiments, the subject has acne vulgaris. In still other embodiments, the subject's skin may be characterized by overgrowth of Staphylococcus aureus, which can be associated with atopic dermatitis. In some embodiments, the subject has psoriasis, and the composition is applied to psoriatic lesions.

In still other embodiments, the topical composition can be employed in a method for facilitating wound healing and/or reducing or preventing fibrosis, as described for example in US 2018/0271953, which is hereby incorporated by reference in its entirety. In some embodiments, the subject has a chronic wound, such as a diabetic ulcer, an amputation wound, or a skin or tissue graft (including a donor site). In some embodiments, the wound is a burn, such as a first- or second-degree burn. In some embodiments, the subject has a sun burn, which can be treated with a topical lotion, gel, or ointment according to this disclosure.

In still other embodiments, the composition is formulated for administration to one or more of the eyes, ears, nasal or sinus cavity, and oral cavity. Compositions according to these embodiments can be useful in a method for treating or preventing inflammation or infection of the eyes of a subject, for example, where the composition is formulated as an eye drop or eye wash (including an eye lid cleaner). In some embodiments, the subject may have dry eye disease, or may have allergic, bacterial, or viral conjunctivitis. Bacterial conjunctivitis can be exacerbated by biofilm formation. In some embodiments, the subject has inflamed ducts of the eye, as may manifest as blepharitis or related condition (e.g., chalazion). In still other embodiments, the composition can be useful for treating or preventing inflammation or infection of the ears of a subject, for example, where the composition formulated as an ear drop. An exemplary condition is an ear infection, such as otitis media. In still other embodiments, the composition can be useful in a method for treating or preventing of nasal or sinus inflammation or infection of a subject, for example, where the composition is formulated as a nasal spray. Exemplary conditions include rhinitis, rhino sinusitis, and post-operative rhinoplasty. In some embodiments, the subject has a sinus infection or recurring sinus infection. In still other embodiments, the subject has an infection or inflammation of the oral cavity, including gingivitis and dental abscess.

In some embodiments, the composition is useful in a method for the treatment of a nucleic acid-related eye disease, as described in U.S. Pat. No. 9,867,871, which is hereby incorporated by reference in its entirety. Such diseases include dry eye disease, lamellar keratitis, contact lens-associated keratitis, endophthalmitis, infectious crystalline keratopathy, ocular cicatricial pemphigoid (OCP), keratoconjunctivitis sicca (KCS), Sjogren syndrome (SS), Sjogren syndrome associated keratoconjunctivitis sicca, non-Sjogren syndrome associated keratoconjunctivitis sicca, keratitis sicca, sicca syndrome, xerophthalmia, tear film disorder, decreased tear production, aqueous tear deficiency (ATD), and meibomian gland dysfunction (MGD).

In still other embodiments, the composition is formulated for pulmonary delivery, such as a powder or solution aerosol. In some embodiments, the composition is formulated for delivery by nebulizer. In various embodiments, the composition is useful in a method for treating or preventing inflammation or infection of the lungs of a patient. In some embodiments, the subject has cystic fibrosis, pneumonia (including ventilator-associated pneumonia, or VAP), bronchitis, asthma, lower respiratory tract infection, acute respiratory distress syndrome (ARDS), acute lung injury (ALI), transfusion-induced lung injury (TRALI), or chronic obstructive pulmonary disease (COPD). In some embodiments, the subject has pulmonary fibrosis (e.g., idiopathic pulmonary fibrosis, or IPF). In still other embodiments, the subject has a chronic or recurring pulmonary infection.

In the case of cystic fibrosis, it is difficult to eradicate or control Pseudomonas aeruginosa infection, which is partially due to the forming of biofilms. By administering the D1L2 enzyme in accordance with this disclosure, for example, by nebulizer, it may be possible to control Pseudomonas infection in cystic fibrosis patients and relieve some overuse of antibiotics. In various embodiments, the D1L2 enzyme provided herein has higher activity in CF lung secretions than PULMOZYME (dornase alfa).

In still other embodiments, the patient may have an indwelling medical device, which tend to favor microbial colonization and biofilm formation. In some embodiments, the D1L2 enzyme or composition comprising the D1L2 enzyme is administered to a patient on mechanical ventilation to avoid or reduce biofilm formation.

The compositions described herein are useful for treating or controlling infection and/or colonization by biofilm microorganisms, including on mammalian tissues as well as on the surface of indwelling medical devices. Pathogens and opportunists that form biofilms include Pseudomonas aeruginosa, Staphylococcus aureus, Staphylococcus epidermidis, Escherichia coli, Klebsiella pneumoniae, Proteus mirabilis, Enterococcus faecalis, Haemophilus influenzae, group A beta-hemolytic streptococci, enteric bacteria, mycobacteria (including non-tuberculous mycobacterium), and Streptococcus pneumoniae. Biofilm formation by these bacteria causes recalcitrance to antibiotics. These pathogens cause the following infections (without limitation): native valve endocarditis (NVE), otitis media (OM), chronic bacterial prostatitis, cystic fibrosis (CF), bloodstream infections, urinary tract infections (optionally associated with biofilm on urinary catheters), prosthetic valve endocarditis (PVE), eye infections (optionally associated with biofilm on contact lenses), and pelvic inflammatory disease (optionally associated with biofilm on intrauterine devices (IUDs)).

As used herein, “treatment” or “treating” or “treated” refers to therapeutic treatment wherein the object is to slow (lessen) an undesired physiological condition, disorder or disease, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. In other embodiments, “treatment” or “treating” or “treated” refers to prophylactic measures, wherein the object is to delay onset of or reduce severity of an undesired physiological condition, disorder or disease, such as, for example is a person who is predisposed to a disease.

As used herein, the term “about” means ±10% of an associated numerical value.

### EXAMPLES

### Recombinant Expression of DNASE1L2 and Extracellular Chromatin-Degrading Activity

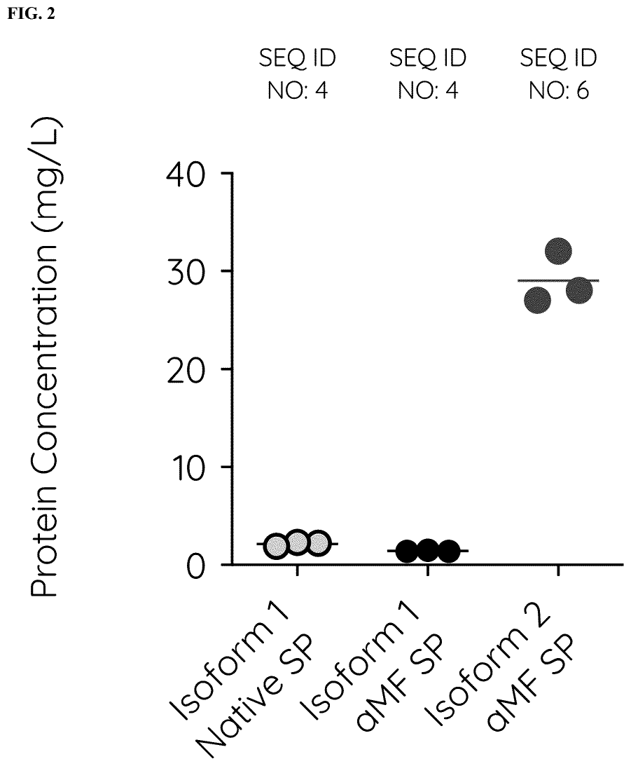

An amino acid sequence alignment of DNASE1L2 isoform 1 and 2 identifies a proline-rich domain, GERAPPLPSRRALTPPPLPAA (SEQ ID NO: 3), in the center of DNASE1L2 isoform 1 (FIG. 1). DNASE1L2 isoform 1 (SEQ ID NO: 1) was cloned and expressed in Pichia pastoris using the native secretory signal peptide. Analysis of culture supernatants detected only negligible amount of DNASE1L2 protein, indicating that DNASE1L2 isoform 1 is not suitable for production in Pichia pastoris (FIG. 2). The native secretory signal peptide was replaced with the alpha-mating factor pre-pro peptide (aMF; SEQ ID NO: 9), which potentiates protein secretion in Pichia pastoris. However, secretion of new aMF-DNASE1L2 isoform 1 construct by Pichia pastoris remained low as no significant amount of target protein could be detected in culture supernatants.

We investigated an expression vector comprising aMF and DNASE1L2 isoform 2 in this study. Strikingly, the new construct was expressed and secreted in a robust manner by Pichia pastoris and substantial amounts of target protein could be detected in culture supernatant (FIG. 2). Thus, the elimination of the PRD enables large scale manufacturing of DNASE1L2 in yeast expression systems, such Pichia pastoris.

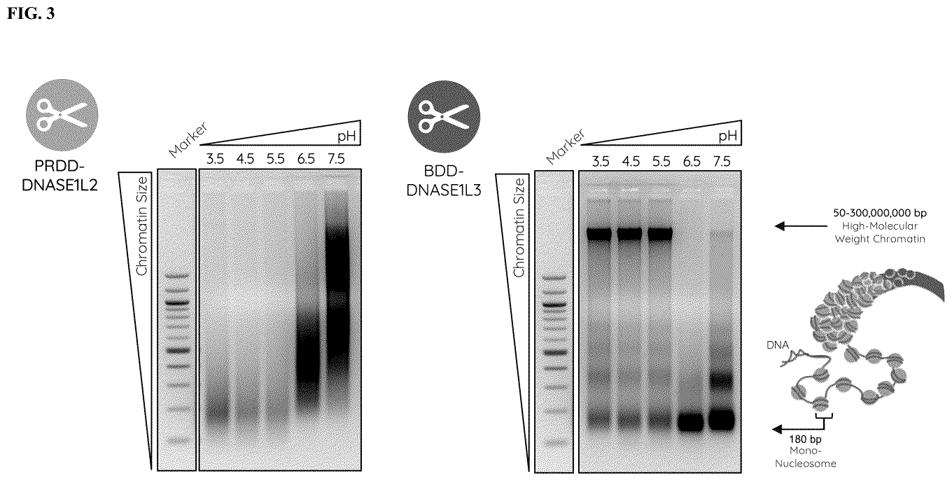

A fermentation at 5L scale was performed and PRDD-DNASE1L2 (SEQ ID NO: 2) was purified using affinity chromatography. The purified protein was tested for enzymatic activity. While previous reports have measured the degradation of purified DNA by DNASE1L2, high-molecular weight chromatin derived from intact nuclei of HEK293 cells was used in this Example as a substrate. A robust chromatin degrading activity of PRDD-DNASE1L2 was observed at pH levels below 6.5, whereas basic domain deleted DNASE1L3 showed optional chromatin degradation at neutral pH (FIG. 3). These data indicate that the deletion of the PRD in DNASE1L2 enables large scale manufacturing of an enzyme that efficiently degrades chromatin in an acidic environment.

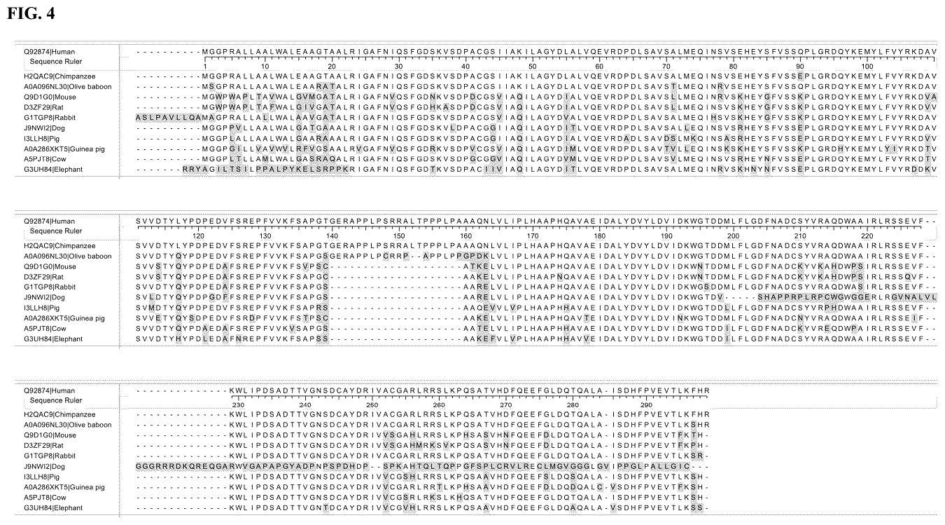

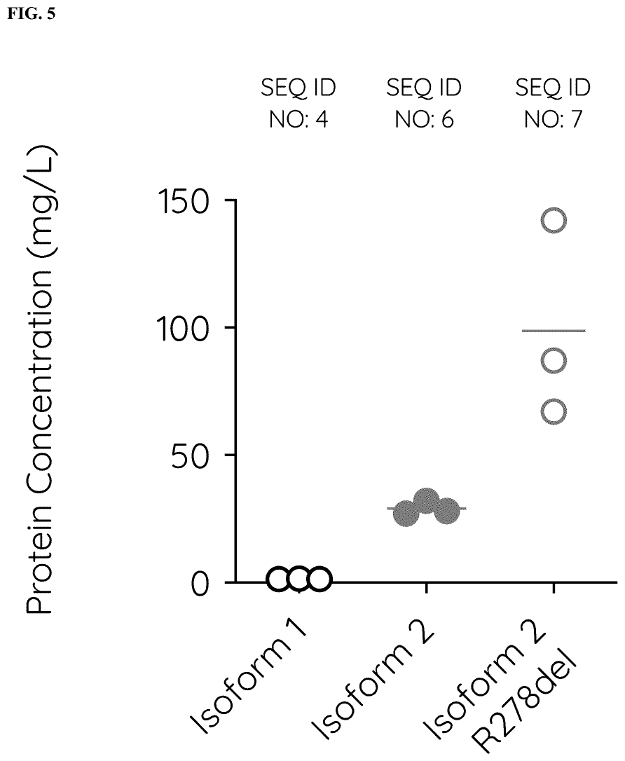

An alignment of DNASE1L2 amino acid sequences from different species in Uniprot indicated that the isoform 1, which comprises a PRD, is expressed in humans, chimpanzees, and baboons, whereas mice, rats, rabbits, dogs, pigs, guinea pigs, cows, and elephants do not. Furthermore, only DNASE1L2 from humans, chimpanzees, and baboons features a C-terminal arginine residue (FIG. 4). Thus, a derivative having the deletion C-terminal arginine residue was studied with hope of finding a DNASE1L2 variant with improved features. Surprisingly, as shown in FIG. 5, the screening of Pichia pastoris clones expressing the DNASE1L2 isoform 2 R278del variant (SEQ ID NO: 7) identified 3 clones with substantially increased production levels, when compared to DNASE1L2 isoform 2 (SEQ ID NO: 6).

PRDD-DNASE1L2 and variants thereof are particularly suitable for degrading extracellular chromatin, including neutrophil extracellular traps (NETs), under acidic conditions, e.g. in acidic sputum of patients with inflammatory lung diseases, such as cystic fibrosis and asthma (Kodric M, Shah AN, Fabbri LM, et al. An investigation of airway acidification in asthma using induced sputum: A study of feasibility and correlation. Am J Respir Crit Care Med. 2007;175(9):905-10; Dropping acid: why is cystic fibrosis mucus abnormal? Eur Respir J. 2018 Dec 6;52(6):1802057) as a well as on the skin, which has naturally an acidic pH.

Recombinant expression of PRDD-DNASE1L2 as a fusion with albumin was also achieved in Pichia pastoris. Albumin was fused at the N-terminus of PRDD-DNASE1L2 through a flexible linker composed of Gly and Ser. In these embodiments, PRDD-DNASE1L2 fusion is secreted by virtue of the human albumin secretory signal peptide (SEQ ID NO:10). This system likewise provides for good expression of PRDD-DNASE1L2.

SEQUENCES
Wild-Type DNASE1L2 Enzymes
SEQ ID NO: 1
Human D1L2 isoform 1, Signal Peptide, Mature Protein, Proline-
Rich Domain
MGGPRALLAALWALEAAGTAALRIGAFNIQSFGDSKVSDPACGSIIAKILAGYDLALVQE
VRDPDLSAVSALMEQINSVSEHEYSFVSSQPLGRDQYKEMYLFVYRKDAVSVVDTYLYPD
PEDVFSREPFVVKFSAPGTGERAPPLPSRRALTPPPLPAAAQNLVLIPLHAAPHQAVAEI
DALYDVYLDVIDKWGTDDMLFLGDFNADCSYVRAQDWAAIRLRSSEVEKWLIPDSADTTV
GNSDCAYDRIVACGARLRRSLKPQSATVHDFQEEFGLDQTQALAISDHFPVEVTLKFHR
SEQ ID NO: 2
Human D1L2 isoform 2, Signal Peptide, Mature Protein:
MGGPRALLAALWALEAAGTAALRIGAFNIQSFGDSKVSDPACGSIIAKILAGYDLALVQE
VRDPDLSAVSALMEQINSVSEHEYSFVSSQPLGRDQYKEMYLFVYRKDAVSVVDTYLYPD
PEDVFSREPFVVKFSAPGTAQNLVLIPLHAAPHQAVAEIDALYDVYLDVIDKWGTDDMLF
LGDFNADCSYVRAQDWAAIRLRSSEVEKWLIPDSADTTVGNSDCAYDRIVACGARLRRSL
KPQSATVHDFQEEFGLDQTQALAISDHFPVEVTLKEHR
Proline-Rich Domain in DNASE1L2, Isoform 1
SEQ ID NO: 3
GERAPPLPSRRALTPPPLPAA
DNASE1L2 Variants
SEQ ID NO: 4
Human D1L2 isoform 1: Mature Protein, Proline-Rich Domain
LRIGAFNIQSFGDSKVSDPACGSIIAKILAGYDLALVQEVRDPDLSAVSALMEQINSVSE
HEYSFVSSQPLGRDQYKEMYLFVYRKDAVSVVDTYLYPDPEDVESREPFVVKFSAPGTGE
RAPPLPSRRALTPPPLPAAAQNLVLIPLHAAPHQAVAEIDALYDVYLDVIDKWGTDDMLF
LGDENADCSYVRAQDWAAIRLRSSEVFKWLIPDSADTTVGNSDCAYDRIVACGARLRRSL
KPQSATVHDFQEEFGLDQTQALAISDHFPVEVTLKFHR
SEQ ID NO: 5
Human D1L2 isoform 1-variant: R299del mutant, Mature Protein,
Proline-Rich Domain
LRIGAFNIQSFGDSKVSDPACGSIIAKILAGYDLALVQEVRDPDLSAVSALMEQINSVSE
HEYSFVSSQPLGRDQYKEMYLFVYRKDAVSVVDTYLYPDPEDVFSREPFVVKFSAPGTGE
RAPPLPSRRALTPPPLPAAAQNLVLIPLHAAPHQAVAEIDALYDVYLDVIDKWGTDDMLF
LGDFNADCSYVRAQDWAAIRLRSSEVFKWLIPDSADTTVGNSDCAYDRIVACGARLRRSL
KPQSATVHDFQEEFGLDQTQALAISDHFPVEVTLKFH
SEQ ID NO: 6
Human D1L2 isoform 2: Mature Protein
LRIGAFNIQSFGDSKVSDPACGSIIAKILAGYDLALVQEVRDPDLSAVSALMEQINSVSE
HEYSFVSSQPLGRDQYKEMYLFVYRKDAVSVVDTYLYPDPEDVFSREPFVVKFSAPGTAQ
NLVLIPLHAAPHQAVAEIDALYDVYLDVIDKWGTDDMLFLGDFNADCSYVRAQDWAAIRL
RSSEVFKWLIPDSADTTVGNSDCAYDRIVACGARLRRSLKPQSATVHDFQEEFGLDQTQA
LAISDHFPVEVTLKFHR
SEQ ID NO: 7
Human D1L2 isoform 2-variant: R278del mutant, Mature Protein
LRIGAFNIQSFGDSKVSDPACGSIIAKILAGYDLALVQEVRDPDLSAVSALMEQINSVSE
HEYSFVSSQPLGRDQYKEMYLFVYRKDAVSVVDTYLYPDPEDVFSREPFVVKFSAPGTAQ
NLVLIPLHAAPHQAVAEIDALYDVYLDVIDKWGTDDMLFLGDENADCSYVRAQDWAAIRL
RSSEVFKWLIPDSADTTVGNSDCAYDRIVACGARLRRSLKPQSATVHDFQEEFGLDQTQA
LAISDHFPVEVTLKFH
Secretory Signal Peptides
SEQ ID NO: 8
D1L2 Signal Peptide
MGGPRALLAALWALEAAGTAA
SEQ ID NO: 9
Alpha mating factor of Pichia pastoris
MRFPSIFTAVLFAASSALAAPVNTTTEDETAQIPAEAVIGYSDLEGDEDVAVLPFSNSTN
NGLLFINTTIASIAAKEEGVS
SEQ ID NO: 10
Human Albumin Secretory Signal Peptide + Propeptide
MKWVTFISLLFLFSSAYSRGVERR
SEQ ID NO: 13
D1 Signal Peptide
MRGMKLLGALLALAALLQGAVS
SEQ ID NO: 14
D1L1 Signal Peptide
MHYPTALLFLILANGAQA
SEQ ID NO: 15
D1L3 Signal Peptide
MSRELAPLLLLLLSIHSALA
Albumin Sequences
SEQ ID NO: 11
Human Serum Albumin (Mature Protein) :
DAHKSEVAHRFKDLGEENFKALVLIAFAQYLQQCPFEDHVKLVNEVTEFAKTCVADESAE
NCDKSLHTLFGDKLCTVATLRETYGEMADCCAKQEPERNECFLQHKDDNPNLPRLVRPEV
DVMCTAFHDNEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEVSKLVTDLTK
VHTECCHGDLLECADDRADLAKYICENQDSISSKLKECCEKPLLEKSHCIAEVENDEMPA
DLPSLAADFVESKDVCKNYAEAKDVFLGMFLYEYARRHPDYSVVLLLRLAKTYETTLEKC
CAAADPHECYAKVFDEFKPLVEEPQNLIKONCELFEQLGEYKFQNALLVRYTKKVPQVST
PTLVEVSRNLGKVGSKCCKHPEAKRMPCAEDYLSVVLNQLCVLHEKTPVSDRVTKCCTES
LVNRRPCFSALEVDETYVPKEFNAETFTFHADICTLSEKERQIKKQTALVELVKHKPKAT
KEQLKAVMDDFAAFVEKCCKADDKETCFAEEGKKLVAASQAALGL
Linker Sequences
SEQ ID NO: 12
Flexible peptide linker:
GGGGSGGGGSGGGGS

1. A method for treating a subject in need of extracellular DNA degradation, extracellular chromatin degradation, extracellular trap (ET) degradation and/or neutrophil extracellular trap (NET) degradation, the method comprising administering a therapeutically effective amount of DNASE1L2 (D1L2) enzyme substantially lacking the Proline Rich Domain (PRD) defined by SEQ ID NO: 3 and/or lacking a C-terminal Arginine residue.

2. The method of claim 1, wherein the D1L2 enzyme substantially lacks the PRD.

3. The method of claim 1, wherein the D1L2 enzyme lacks the C-terminal Arginine residue.

4. The method of claim 1, wherein the D1L2 enzyme substantially lacks the PRD and lacks the C-terminal Arginine residue.

5. The method of any one of claims 1 to 4, wherein the D1L2 enzyme is PEGylated.

6. The method of claim 5, wherein the D1L2 enzyme is PEGylated at a one or more cysteines.

7. The method of claim 5, wherein the D1L2 enzyme is PEGylated at primary amines.

8. The method of any one of claims 1 to 7, wherein the D1L2 is fused to a carrier protein at the N- or C-terminus.

9. The method of claim 8, wherein the carrier protein is an albumin amino acid sequence.

10. The method of claim 9, wherein the albumin amino acid sequence fused to the N-terminus of D1L2 optionally through a peptide linker.

11. The method of any one of claims 1 to 10, wherein the subject has accumulated extracellular DNA, extracellular chromatin, extracellular traps (ETs) and/or neutrophil extracellular traps (NETs) in an acidic environment.

12. The method of claim 11, wherein the D1L2 enzyme is applied to a tissue environment having a pH of less than 6.5, or having a pH of about 5.5 or less, or having a pH of about 5.0 or less.

13. The method of claim 12, wherein the subject has a pulmonary indication.

14. The method of claim 13, wherein the subject has cystic fibrosis, pneumonia, acute respiratory distress syndrome (ARDS), acute lung injury (ALI), transfusion-induced lung injury (TRALI), asthma, Chronic Obstructive Pulmonary Disorder (COPD), pulmonary fibrosis, or pulmonary infection, and the D1L2 is administered to the lungs.

15. The method of claim 12, wherein the subject has a dermatological condition, and the D1L2 is administered to the subject's skin.

16. The method of claim 15, wherein the D1L2 is applied to parakeratotic lesions.

17. The method of claim 15, wherein the subject has psoriasis, dermatitis, epidermolysis bullosa, or an allergic reaction.

18. The method of claim 15, wherein the subject has acne vulgaris.

19. The method of claim 15, wherein the subject has overgrowth of Staphylococcus aureus.

20. The method of claim 11, wherein the D1L2 is applied to a slow healing wound.

21. The method of claim 20, wherein the wound is a chronic wound, which is optionally a diabetic ulcer, an amputation wound, or a skin or tissue graft.

22. The method of claim 20, wherein the wound is a burn.

23. The method of claim 11, wherein the D1L2 enzyme is applied to the eyes of the subject, to treat or prevent inflammation or infection of the eyes.

24. The method of claim 23, wherein the subject has dry eye disease.

25. The method of claim 23, wherein the subject has allergic, bacterial, or viral conjunctivitis.

26. The method of claim 11, wherein the D1L2 enzyme is applied to the ears of the subject, to treat or prevent inflammation or infection of the ears.

27. The method of claim 26, wherein the subject has otitis media.

28. The method of claim 11, wherein the D1L2 enzyme is applied to the nose or sinus cavity of the subject, to treat or prevent nasal or sinus inflammation or infection.

29. The method of any one of claims 1 to 28, wherein the D1L2 enzyme comprises an amino acid sequence having at least about 80% sequence identity, or at least about 85%, at least about 90%, or at least about 95%, or at least about 97% sequence identity to the enzyme defined by SEQ ID NO: 5 or SEQ ID NO: 7.

30. The method of claim 29, wherein the D1L2 enzyme comprises the amino acid sequence of SEQ ID NO: 7.

31. The method of any one of claims 1 to 30, wherein the D1L2 enzyme is produced in a non-mammalian expression system, which is optionally a yeast expression system such as Pichia pastoris.

32. A method for production of a recombinant D1L2 enzyme, the method comprising: culturing a non-mammalian expression host cell encoding a D1L2 enzyme substantially lacking the Proline Rich Domain (PRD) defined by SEQ ID NO: 8 and/or lacking a C-terminal Arginine, and comprising a signal peptide; and recovering the D1L2 enzyme from the culture media.

33. The method of claim 32, wherein the expression host cell is a eukaryotic cell, optionally selected from a yeast cell, an insect cell, and a plant cell.

34. The method of claim 33, wherein the expression host cell is a yeast cell, optionally selected from Pichia pastoris, Saccharomyces cerevisiae, and Yarrowia lipolytica.

35. The method of claim 32, wherein the expression host cell is a bacterial host cell, which is optionally E. coli.

36. The method of any one of claims 32 to 35, wherein the D1L2 enzyme lacks at least about 10 amino acids of the PRD (SEQ ID NO: 8).

37. The method of claim 36, wherein the D1L2 enzyme lacks at least about 15 amino acids of the PRD (SEQ ID NO: 8).

38. The method of claim 36, wherein the D1L2 enzyme lacks at least about 18 amino acids of the PRD (SEQ ID NO: 8).

39. The method of claim 36, wherein the D1L2 enzyme lacks the full PRD (SEQ ID NO: 8).

40. The method of any one of claims 32 to 39, wherein the D1L2 enzyme comprises an amino acid sequence having at least about 80% sequence identity, or at least about 85% sequence identity, or at least about 90% sequence identity, or at least about 95% sequence identity, or at least about 97% sequence identity to the enzyme defined by SEQ ID NO: 5 or SEQ ID NO: 7.

41. The method of claim 40, wherein the D1L2 enzyme comprises the amino acid sequence of SEQ ID NO: 7.

42. The method of any one of claims 32 to 41, wherein the signal peptide is from a wild-type human DNase.

43. The method of claim 42, wherein the human wild-type DNase is D1, D1L1, D1L2, or D1L3.

44. The method of any one of claims 32 to 41, wherein the signal peptide is a non-native signal peptide for a human DNase enzyme, and which is optionally α Mating Factor or Human Albumin Secretory Signal Peptide.

45. The method of any one of claims 32 to 44, wherein the D1L2 is fused to a carrier protein at the N- or C-terminus.

46. The method of claim 45, wherein the carrier protein is an albumin amino acid sequence.

47. The method of claim 46, wherein the albumin amino acid sequence fused to the N-terminus of D1L2 optionally through a peptide linker.

48. The method of any one of claims 32 to 47, wherein the expression results in at least about 100 mg/L of the D1L2 enzyme in a batch fermentation culture of at least 1000 L.

49. The method of claim 48, wherein the expression results in at least about 250 mg/L of the D1L2 enzyme in a batch fermentation culture of at least 1000 L.

50. The method of any one of claims 32 to 49, wherein the recombinant D1L2 enzyme is not glycosylated.

51. The method of any one of claims 48 to 50, further comprising, PEGylating the D1L2 enzyme.

52. The method of claim 51, wherein the D1L2 enzyme is PEGylated at one or more cysteines.

53. The method of claim 51, wherein the D1L2 enzyme is PEGylated at primary amines.

54. A recombinant D1L2 enzyme produced according to the method of any one of claims 32 to 53.

55. A polynucleotide encoding the D1L2 enzyme of claim 54, wherein the polynucleotide is codon optimized for expression in the non-mammalian expression system, which is optionally Pichia pastoris.

56. The polynucleotide of claim 55, wherein the polynucleotide comprises a plasmid vector.

57. A non-mammalian host cell comprising the vector of claim 56 and expressing the D1L2 enzyme.

58. A pharmaceutical composition for topical or local administration, comprising:
an effective amount of the recombinant D1L2 enzyme of claim 54, or a D1L2 enzyme substantially lacking the PRD of SEQ ID NO: 3 and/or lacking a C-terminal Arginine residue, and a pharmaceutically acceptable carrier.

59. The pharmaceutical composition of claim 58, wherein the D1L2 enzyme comprises an amino acid sequence having at least about 80% sequence identity, or at least about 85% sequence identity, or at least about 90% sequence identity, or at least about 95% sequence identity, or at least about 97% sequence identity to the enzyme defined by SEQ ID NO: 7.

60. The pharmaceutical composition of claim 59, wherein the D1L2 enzyme lacks the PRD of SEQ ID NO: 3 and lacks a C-terminal Arginine residue.

61. The pharmaceutical composition of claim 60, wherein the D1L2 enzyme comprises the amino acid sequence of SEQ ID NO: 7.

62. The pharmaceutical composition of any one of claims 58 to 61, wherein the recombinant D1L2 enzyme is not glycosylated.

63. The pharmaceutical composition of claim 62, wherein the D1L2 enzyme is PEGylated.

64. The pharmaceutical composition of claim 63, wherein the D1L2 enzyme is PEGylated at one or more cysteines.

65. The pharmaceutical composition of claim 63, wherein the D1L2 enzyme is PEGylated at primary amines.

66. The pharmaceutical composition of any one of claims 58 to 65, wherein the D1L2 enzyme is fused to a carrier protein at the N- or C-terminus.

67. The pharmaceutical composition of claim 66, wherein the carrier protein is an albumin amino acid sequence.

68. The pharmaceutical composition of claim 67, wherein the albumin amino acid sequence fused to the N-terminus of D1L2 optionally through a peptide linker.

69. The pharmaceutical composition of any one of claims 58 to 68, formulated for topical administration.

70. The pharmaceutical composition of claim 69, wherein the composition is a cream, lotion, gel, foam, spray, ointment, mouthwash, soap, or shampoo.

71. The pharmaceutical composition of claim 69, wherein the composition is formulated for administration to one or more of the eyes, ears, nasal or sinus cavity, and oral cavity.

72. The pharmaceutical composition of any one of claims 58 to 68, wherein the composition is formulated for pulmonary delivery.

73. The pharmaceutical composition of claim 72, wherein the composition is a powder aerosol or solution aerosol, or formulated for delivery by nebulizer.

74. A method of treating or preventing infection and/or inflammation of the skin, comprising, administering the pharmaceutical composition of claims 58 to 70 to affected areas of a subject's skin.

75. The method of claim 74, wherein the affected area comprises parakeratotic lesions.

76. The method of claim 44 or 75, wherein the subject has psoriasis, dermatitis, epidermolysis bullosa, or an allergic reaction.

77. The method of claim 74, wherein the subject has acne vulgaris.

78. The method of claim 44, wherein the subject has overgrowth of Staphylococcus aureus.

79. A method for facilitating wound healing, comprising, administering the pharmaceutical composition of claim 44 to a wound of a subject.

80. The method of claim 79, wherein the wound is a chronic wound, which is optionally a diabetic ulcer, an amputation wound, or a skin or tissue graft.

81. The method of claim 79, wherein the wound is a burn.

82. A method for treating or preventing inflammation or infection of the eyes of a subject, the method comprising administering the composition of claim 71 to a subject in need, the composition formulated as an eye drop or eye wash.

83. The method of claim 82, wherein the subject has dry eye disease.

84. The method of claim 82, wherein the subject has allergic, bacterial, or viral conjunctivitis.

85. A method for treating or preventing inflammation or infection of the ears of a subject, the method comprising administering the composition of claim 71 to a subject in need, the composition formulated as an ear drop.

86. The method of claim 85, wherein the subject has otitis media.

87. A method for treating or preventing of nasal or sinus inflammation or infection of a subject, the method comprising administering the composition of claim 71 to a subject in need, the composition formulated as a nasal spray.

88. The method of claim 87, wherein the subject has a sinus infection.

89. A method for treating or preventing inflammation or infection of the lungs of a patient, comprising, administering the composition of claim 72 or 73 to a subject in need.

90. The method of claim 89, wherein the subject has cystic fibrosis, pneumonia, acute respiratory distress syndrome (ARDS), acute lung injury (ALI), transfusion-induced lung injury (TRALI), asthma, Chronic Obstructive Pulmonary Disorder (COPD), pulmonary fibrosis, or pulmonary infection.

91. The method of claim 99, wherein the subject has a chronic or recurring pulmonary infection.


SEQUENCE LISTING (15 sequences)

SEQ ID NO: 1 - protein, 299 aa, Homo sapiens
MGGPRALLAALWALEAAGTAALRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTGERAPPLPSRRALTPPPLPAAAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 2 - protein, 278 aa, Homo sapiens
MGGPRALLAALWALEAAGTAALRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 3 - protein, 21 aa, Artificial Sequence
  Synthetic Sequence
GERAPPLPSRRALTPPPLPAA

SEQ ID NO: 4 - protein, 278 aa, Homo sapiens
LRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTGERAPPLPSRRALTPPPLPAAAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 5 - protein, 277 aa, Homo sapiens
LRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTGERAPPLPSRRALTPPPLPAAAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFH

SEQ ID NO: 6 - protein, 257 aa, Homo sapiens
LRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 7 - protein, 256 aa, Homo sapiens
LRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFH

SEQ ID NO: 8 - protein, 21 aa, Artificial Sequence
  Synthetic Sequence
MGGPRALLAALWALEAAGTAA

SEQ ID NO: 9 - protein, 81 aa, Artificial Sequence
  Synthetic Sequence
MRFPSIFTAVLFAASSALAAPVNTTTEDETAQIPAEAVIGYSDLEGDFDVAVLPFSNSTNNGLLFINTTIASIAAKEEGVS

SEQ ID NO: 10 - protein, 24 aa, Artificial Sequence
  Synthetic Sequence
MKWVTFISLLFLFSSAYSRGVFRR

SEQ ID NO: 11 - protein, 585 aa, Homo sapiens [sequence omitted]

SEQ ID NO: 12 - protein, 15 aa, Artificial Sequence
  Synthetic Sequence
GGGGSGGGGSGGGGS

SEQ ID NO: 13 - protein, 22 aa, Artificial Sequence
  Synthetic Sequence
MRGMKLLGALLALAALLQGAVS

SEQ ID NO: 14 - protein, 18 aa, Artificial Sequence
  Synthetic Sequence
MHYPTALLFLILANGAQA

SEQ ID NO: 15 - protein, 20 aa, Artificial Sequence
  Synthetic Sequence
MSRELAPLLLLLLSIHSALA